(19) United States
(12) Patent Application Publication
(10) Pub. No.: US 2024/0408321 A1
(43) Pub. Date: Dec. 12, 2024

(54) DEAD VOLUME REDUCING CONNECTOR

(71) Applicant: AKTIVAX, INC., Broomfield, CO (US)

(72) Inventors: Amir Genosar, Broomfield, CO (US); Greg Langley, Broomfield, CO (US)

(21) Appl. No.: 18/701,529

(22) PCT Filed: Oct. 21, 2022
(86) PCT No.: PCT/US2022/078529

§ 371 Date: Apr. 15, 2024

Related U.S. Application Data
(60) Provisional application No. 63/270,364, filed on Oct. 21, 2021.
(60) Provisional application No. 63/304,423, filed on Jan. 28, 2022.

(51) Int. Cl. A61M 5/32; A61M 5/34

(52) U.S. Cl. CPC A61M 5/3293; A61M 5/347

(57) ABSTRACT

A small bore medical device connector is provided. The small bore medical device connector includes a female connector comprising a fluid path and a receptacle configured to accept a male Luer lock connector protrusion extending from a male Luer lock connector. The female connector is configured for threaded engagement to the male Luer lock connector. The male Luer lock connector protrusion includes a standard Luer tapered protrusion and a flow channel therewithin. The male Luer lock connector terminates at a tip disposed at the distal portion. The tip includes an opening to the flow channel. When the female connector is engaged with the male Luer lock connector, a distal aspect of the tip contactingly engages a distal engagement surface in the receptacle to establish a fluid-tight seal and to establish fluid communication through a combined fluid pathway including the fluid path and the flow channel.

### CROSS-REFERENCE TO RELATED APPLICATION(S)

The present application claims priority to U.S. Provisional Patent Application Ser. No. 63/270,364, filed Oct. 21, 2021, and entitled “Low Dead-Volume Female Connector for Standard Male Luer Lock Connections,” and to U.S. Provisional Patent Application Ser. No. 63/304,423, filed Jan. 28, 2022, and entitled “Low Dead-Volume Female Connector,” which are incorporated by reference herein in their entirety. In the event that definitions or descriptive terms used in this or any other patent incorporated by reference conflict with definitions or descriptive terms used in the present application, the use in the present application will control.

### BACKGROUND

Pharmaceutical companies may now reduce or minimize drug delivery hindrances, such as inaccuracies and end-user errors, by encapsulating pharmaceutical agents and delivery devices into one discrete package. By eliminating end-user interferences, the delivery of the pharmaceutical agent is more tightly controlled and thus can be more accurate and efficient. The increased accuracy may also lead to reduced dead space within a syringe and needle pairing and creates dead space volume of an injectable drug. Dead space volume refers to the amount of fluid remaining within the syringe and needle after an injection is completed. Low dead volume connectors may reduce product loss, may lead to reduced overfill, or may prevent superfluously extracting doses of a pharmaceutical drug.

### SUMMARY

The described technology provides a small bore medical device connector. The small bore medical device connector includes a female connector comprising a fluid path and a receptacle configured to accept a male Luer lock connector protrusion extending from a male Luer lock connector. The female connector is configured for threaded engagement to the male Luer lock connector. The male Luer lock connector protrusion includes a standard Luer tapered protrusion and a flow channel therewithin. The male Luer lock connector terminates at a tip disposed at the distal portion. The tip includes an opening to the flow channel. When the female connector is engaged with the male Luer lock connector, a distal aspect of the tip contactingly engages a distal engagement surface in the receptacle to establish a fluid-tight seal and to establish fluid communication through a combined fluid pathway including the fluid path and the flow channel.

This summary is provided to introduce a selection of concepts in a simplified form that is further described below in the Detailed Description. This summary is not intended to identify key features or essential features of the claimed subject matter, nor is it intended to be used to limit the scope of the claimed subject matter.

Other implementations are also described and recited herein.

### BRIEF DESCRIPTIONS OF THE DRAWINGS

Features of examples of the present disclosure will become apparent by reference to the following detailed description and drawings, in which like reference numerals correspond to similar (e.g., analogous), though perhaps not identical, components. For the sake of brevity, reference numerals or features having a previously described function may or may not be described in connection withother drawings in which they appear.

FIG. 1a is a schematic illustration of an example female connector in a confronting position with a Luer Lock syringe.

FIG. 1b illustrates a partial view of the example female connector of FIG. 1a in a first configuration.

FIG. 1c is a schematic illustration of a cross-section view of the example of FIG. 1a in which a seal is configured to interface with the smaller diameter of the tip surface of the male Luer connector.

FIG. 1d is a schematic illustration of a cross-section view of the example of FIG. 1a in a second configuration wherein the syringe in the male Luer connector of the syringe is joined with the female connector.

FIG. 2a is a schematic illustration of an example female connector with a piercing member in a first position.

FIG. 2b is a schematic illustration of an example female connector with a piercing member mated with a male Luer lock connector, wherein the piercing member is in a second position.

FIG. 3a is a schematic illustration of an example female connector comprising a plunger, wherein the interface between the female connector and the plunger form a valve, and the female is in a first configuration wherein the valve is closed.

FIG. 3b is a schematic illustration of an example female connector of FIG. 3a in a second configuration wherein the female connector is mated with a male Luer lock connector, and the valve is open.

FIG. 4 is a schematic illustration of an example female connector mated with a male Luerlock connector, wherein the seal interfaces with the peripheral region of the tip surface of the male Luer connector.

FIG. 5a is a schematic illustration of an example female connector mated with a maleLuer lock connector, wherein the seal comprises a circular ridge feature embedded in the receptacle of the female connector.

FIG. 5b is an enlarged view of an example female connector of FIG. 5a.

FIG. 6 is a schematic illustration of an example female connector comprising a piercing member and a stationary seal that is configured to interface with the larger diameter of the tip surface of a male Luer connector.

FIG. 7 is a schematic illustration of an example female connector comprising a plug.

FIG. 8 is a schematic illustration of an example female connector comprising a retraction spring to form a valve. The retraction spring biases the plunger to the first position to keep the valve closed by default.

FIG. 9a is a schematic illustration of an example female connector comprising an auto-disable mechanism.

FIG. 9b is an exploded view of an example female connector comprising an auto-disable mechanism.

FIG. 10a is an illustration of an example female connector comprising an independent fluid venting pathway comprising an extended bore along the central shaft.

FIG. 10b illustrates the example female connector of FIG. 10a in the joined state with a corresponding male connector.

FIG. 11a is an illustration of an example female connector comprising an independent venting pathway comprising at least one separate bore in the connector body.

FIG. 11b illustrates the female connector of FIG. 11a in the joined state with a corresponding male connector.

FIG. 12a illustrates an external perspective view of an example medical connector.

FIG. 12b illustrates a cross-sectional view of an example medical connector prior to engagement of a male Luer connector and a female connector.

FIG. 12c illustrates a cross-sectional view of an example medical connector after engagement of a male Luer connector and a female connector.

FIG. 12d illustrates a cross-sectional view of an example medical connector after engagement of a male Luer connector and a female connector and after a piercing element is removed.

### DETAILED DESCRIPTIONS

The International Organization for Standardization (ISO) has published ISO 80369-7:2021, which specifies general requirements for small-bore connectors. Small-bore connectors convey liquids or gases in healthcare applications. These small-bore connectors are used in medical devices or accessories intended for use with a patient. Therefore, compliance with ISO Standard 80369 further ensures connectors are specified to mate correctly with one another and create a closed surface.

The Luer taper set forth in ISO 80369-7:2021 describes both the Luer slip and Luer lock configurations. Generally, a Luer connector refers to a small-bore connector that contains a conical mating surface with a 6% (Luer) taper intended for use in intravascular or hypodermic applications of medical devices and related accessories. The Luer slip configuration may include a smooth-sided conical entrance without a threaded lock system. The Luer slip configuration may use tension to hold a needle in position. By contrast, the Luer lock configuration may include a threaded conical entrance designed to lock another component in place via the threads. A design feature of a male-to-female Luer connector is that the depth of the female Luer receptacle is such that the tip of the male Luer connector does not reach the base of the female connector receptacle when engaged. Accordingly, the Luer receptacle may not form a hermetic or fluid-tight seal with the male Luer connector at the base. Rather, as the male Luer connector is inserted into the female receptacle, its outside wall may compress against the Luer tapered interior walls of the receptacle to create a hermetic or fluid-tight seal.

Because of the 6% taper angle of the Luer seal surface, the axial position of the male Luer relative to the female receptacle, when engaged, varies due to manufacturing tolerances and may not be predictable. The 6% taper angle means that for each 0.1 variation in the taper diameter (0.05 radii), the location of the male Luer relative to the female Luer varies by about 0.3 mm. This can cause variations in dead space volume between the tip of the male Luer and the base of the female Luer receptacle in equipment produced by different manufacturers. This dead space volume may affect dose accuracy, may cause dose wastage, and can potentially be a trap for undesirable air bubbles.

At the present time, low dead volume syringes and needles may reduce the dead space volume. Fixed needle syringes may also reduce the dead space volume, but the fixed needle syringes may present challenges with injection and are not suitable for all injections.

This disclosure describes a female connector adaptable to mate with a male Luer lock connector per ISO Standard 80369-7:2021. The male Luer connector may include a Luer protrusion, including a 6% tapered cylindrical wall surface and a tip surface at the smaller diameter end of the cylindrical wall surface, generally perpendicular to the tapered wall surface. The female connector creates a fluid-tight seal with the male Luer lock connector, such that the tip of the male Luer connector may bottom out (e.g., contactingly engage) in a predictable and consistent way against a seal at the bottom of the female connector's receptacle, and decreases the dead space volume of the syringe.

In an embodiment, the female connector may include a body comprising a proximal end including a receptacle configured to receive a male Luer tip, radial protrusions extending from the proximal end configured to communicate with the Luer lock thread and lock the female connector and the male Luer connector in place. In implementations, the female connector may include a seal disposed in the receptacle configured to establish a fluid-tight seal with the tip surface of the male Luer lock connector.

The receptacle may comprise a geometry such that when the male Luer lock connector is engaged, the Luer lock is tightened against the protrusions, and the tip surface of the male Luer lock connector bottoms out against and physically engages (e.g., touches) the seal to form a fluid-tight seal. The male Luer protrusion can be a fluid communication feature of a syringe, a connector, an adapter, a coupler, an infusion Y-site, an infusion bag, a tubing, a pump, a cap, or equivalent fluidic devices.

Drug delivery technologies may decrease the dead space volume of the delivery device. The reduction of dead space volume may lead to additional dosages and may reduce wasted product by enabling further extraction of the product. The reduction of dead space volume may also provide better accuracy in dosing.

The female connector may attach to the male Luer connector of the syringe, attaching via threads in the Luer lock configuration. Examples of the connector may include a piercing member. The piercing member may be configured such that when the female connector is not attached to the male Luer connector, the piercing member is in a first position. The piercing member may then be moved into a second position by the attachment of the male Luer connector. The piercing member may be configured to break a seal at the distal end of the female connector.

Throughout this disclosure, the terms “proximal” and “distal” are used to orient the connector and the configuration of the connector. As used herein, the term “proximal” refers to a direction towards the position of the connector closest to the syringe and the Luer taper. As used herein, the term “distal” refers to a direction toward the position of the connector further from the syringe.

Throughout this disclosure, the term “thread” or “threads” may refer to the Luer lock configuration threads, as defined in ISO 80369-7:2021.

Throughout this disclosure, the term “perpendicular” refers to an orientation that is perpendicular to another surface. In some cases, the term “generally perpendicular” may refer to a configuration in which a surface may be at a slope or angle and may not form a perpendicular configuration with another surface. In these cases, perpendicularity is implied, referring to the axis of the conical shape and/or the surface of the conical shape.

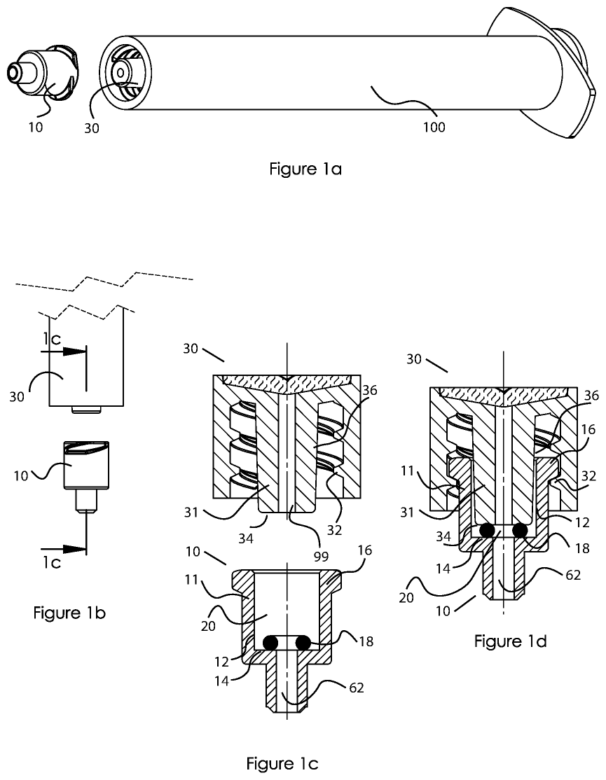

Referring to FIGS. 1a-1c, a female connector 10 may be configured to mate with a male Luer connector 30 (e.g., a male Luer lock connector or a male Luer slip connector). Specifically, FIG. 1a is a schematic illustration of an example female connector in a confronting position with a Luer lock syringe. In the example illustrated in FIG. 1a, the male Luer connector 30 may be a component of a syringe 100. As described with respect to the implementations presented herein, the female connector 10 may engage with (e.g., threadedly engage with or otherwise couple to) the male Luer connector 30 to form a medical connector (e.g., a small bore medical connector).

FIG. 1b illustrates a partial view of the example female connector of FIG. 1a in a first configuration. In FIG. 1b, the horizontal arrows labeled “1c” illustrate the locations of the cross-section planes of the female connector 10) and the male Luer connector 30 pictured in subsequent figures. The female connector 10 may be formed from Polypropylene (PP), Polyethylene (PE), Polycarbonate (PC), Ethylene Vinyl Acetate (EVA), stainless steel, or other materials known inthe art. In other examples, the connector may be formed from other suitable materials and is not limited to the examples provided herein.

FIG. 1c is a schematic illustration of a cross-section view of the example of FIG. 1a in which a seal is configured to interface with the smaller diameter of the tip surface 34 of the male Luer connector 30. The female connector 10 may comprise a connector body 11, including a receptacle 20 configured to accept a Luer protrusion 31 (e.g., a male Luer lock connector protrusion that extends from a male Luer lock connector) of the male Luer connector 30. The receptacle 20 may include a recessed volume defined by an interior wall 12 and a distal engagement surface 14 or base surface. The Luer protrusion 31 may include a standard Luer tapered protrusion that extends continuously between a proximal portion and a distal portion of the male Luer lock connector protrusion. The Luer protrusion 31 may include a flow channel 99 therewithin. The female connector 10 may threadedly engage the male Luer connector 30 to form a medical device connector (e.g., a small-bore medical device connector).

The interior wall 12 of the receptacle 20 may be substantially cylindrical or conical in shape and/or may be tapered. In implementations, the slope of the taper may form a conical shape that is larger at the proximal end of the receptacle 20 of the female connector 10 and narrower at the distal end of the receptacle 20 of the female connector 10. In an embodiment, the interior wall 12 may not taper, and the interior diameter of the receptacle may be the same at the proximal end and the distal end of the receptacle 20.

The receptacle 20 may include a distal engagement surface 14 at the distal end of the receptacle 20. In various implementations, the distal engagement surface 14 can alternatively be referred to as a base surface or bottom surface, provided the base surface of the bottom surface engages the tip of the Luer protrusion 31. In an implementation, the distal engagement surface 14 may be generally or substantially perpendicular to the interior wall 12. The distal engagement surface 14 may, additionally or alternatively, be substantially perpendicular to a central longitudinal axis of the Luer protrusion 31. The interior wall 12 and the distal engagement surface 14 of the female connector 10 together form the receptacle 20. In an implementation, the distal engagement surface 14 is a shelf (e.g., a substantially planar shelf, a flat shelf, a sloping shelf, or a shelf with protrusions extending toward the male Luer connector 30 when engaged) relative to the interior wall 12 and a fluid path 62 in the female connector 10.

The receptacle 20 may be configured to accept a male Luer connector 30 at the proximal end of the female connector 10. The receptacle 20 may have radial protrusions 16, also referred to in the art as ‘lugs.’ extending outwardly from a proximal portion of the exterior of the female connector 10 (e.g., the exterior of the receptacle 20) configured to communicate with the male Luer connector 30 (e.g., by threaded engagement). The radial protrusions 16 may be elements of an external threading engagement portion of the female connector 10. The external threading engagement portion of the female connector 10 may be positioned externally of the receptacle 20. The radial protrusions 16 may have a tilt angle that accepts the lock threads 32 of the male Luer connector 30.

The lock threads 32 may be elements of an interior threading portion of the male Luer connector 30. The interior threading portion may face or oppose the Luer protrusion 31. The male Luer connector 30 may include a Luer protrusion 31, including a tapered protrusion 36 (e.g., a standard Luer tapered protrusion with a tapered surface) and a tip surface 34 at the smaller diameter end of the cylindrical wall surface generally perpendicular to the tapered wall surface. The tip surface 34 may distally terminate with a distal aspect of the tip (e.g., the distal-most part of the Luer protrusion 31). In an implementation, the distal aspect of the tip and/or the tip surface 34 faces the distal engagement surface 14. In an implementation, the distal aspect of the tip is configured to contactingly engage the distal engagement surface 14 (e.g., the tip contacts the distal engagement surface 14). The tip may include an opening to the flow channel 99. The lock threads 32 of the male Luer connector 30, and the taper of the tapered protrusion 36 of the Luer protrusion 31 may be as described in ISO 80369-7:2021, which is incorporated by reference herein. The lock threads 32 may be standard threads according to the ISO 80369-7:2021 standard.

The female connector 10 may comprise a fluid path 62 at the distal end in fluid communication with the receptacle 20. In an implementation, the distal aspect of the tip and/or the tip surface 34 engages the distal engagement surface 14 to establish a fluid-tight seal and to establish fluid communication through a combined fluid pathway. The combined fluid pathway may include the fluid path 62 of the female connector and a flow channel 99 of the male Luer lock connector.

The receptacle 20 may be configured to accept a Luer protrusion 31 of the male Luer connector 30 (e.g., a male Luer lock connector or a male Luer slip connector). The receptacle 20 may have a depth that is defined from the proximal end of the connector body 11 and the distal engagement surface 14. The distal engagement surface 14 may represent a shelf portion defined relative to the interior wall 12 and the fluid path 62. In an implementation, the distal engagement surface 14 is substantially (or at least partially) perpendicular to the direction of advancement of the male Luer connector 30 illustrated from FIG. 1c to FIG. 1d. The direction of advancement may be along a longitudinal axis of the male Luer connector 30. The receptacle may have a depth of about 3.5 millimeters (mm) to about 7 mm. The depth of the receptacle 20 may be less than 7.5 mm (e.g., from a proximal end of the receptacle 20 to the distal engagement surface 14 or a distal engagement surface), which may correspond to the minimum depth of a standard female Luer connector taper, and more than 3.2 mm, which is the distance from the bottom tip of a standard male Luer connector to the bottom of the first complete thread profile, as defined by ISO 80369-7:2021. For example, the maximum penetration depth of the Luer protrusion within the receptacle may be less than 7.5 mm during the engagement. The receptacle may therefore be sufficiently deep to ensure engagement of the radial protrusions with the lock threads 32, and sufficiently shallow to ensure that the tip surface 34 of the Luer protrusion 31 can bottom out against the distal engagement surface 14. In an implementation, a geometry of the receptacle 20 is configured such that when the male Luer connector 30 is engaged, the distal aspect of the tip of the male Luer connector 30 bottoms out against the distal engagement surface 14. In an implementation, a distance remains between the interior wall 12 and the Luer protrusion 31 at all axial positions of a longitudinal axis of the Luer protrusion 31 when the distal aspect of the tip contactingly engages the distal engagement surface 14.

In an implementation, a sealing 18 (e.g., a sealing member) may be disposed in the receptacle and be configured to establish a fluid-tight seal between the tip surface 34 of the Luer protrusion 31 and the receptacle 20 (e.g., to establish fluid communication between the fluid path 62 and the flow channel 99. In such embodiments, the tip surface 34 bottoms out against the seal 18. In an implementation, the receptacle 20 may be wider (e.g., have a perimeter or circumference greater) than 4.477 mm. The dimensions and geometry of the receptacle 20 may be adapted to prevent a fluid seal between the tapered protrusion of the Luer protrusion 31 and the receptacle 20. In an implementation, the seal 18 is configured to establish a fluid-tight seal when the distal aspect of the tip bottoms out against the distal engagement surface 14. The geometry of the receptacle 20 may be adapted to form a fluid-tight seal between the distal aspect of the tip and the receptacle 20 using the seal 18.

FIG. 1d is a schematic illustration of a cross-section view of the example of FIG. 1a in a second configuration wherein the syringe in the male Luer connector of the syringe is joined with the female connector. The female connector 10 may be engaged with the male Luer connector 30. The Luer protrusion 31 may be inserted into the receptacle 20 at the proximal end of the female connector 10, and secured via the lock threads 32. The radial protrusions 16 of the female connector 10 may mate with the lock threads 32 (e.g., Luer lock threads) of the male Luer connector 30. The tip surface 34 of the Luer protrusion 31 may form a seal 18 against the distal engagement surface 14 of the female connector 10. The seal 18 may form in the receptacle 20 at the distal end of the female connector 10. As can be seen, the taper of the tapered protrusion 36 of the Luer protrusion 31 may be configured to not seal against the interior wall 12 of the female connector 10.

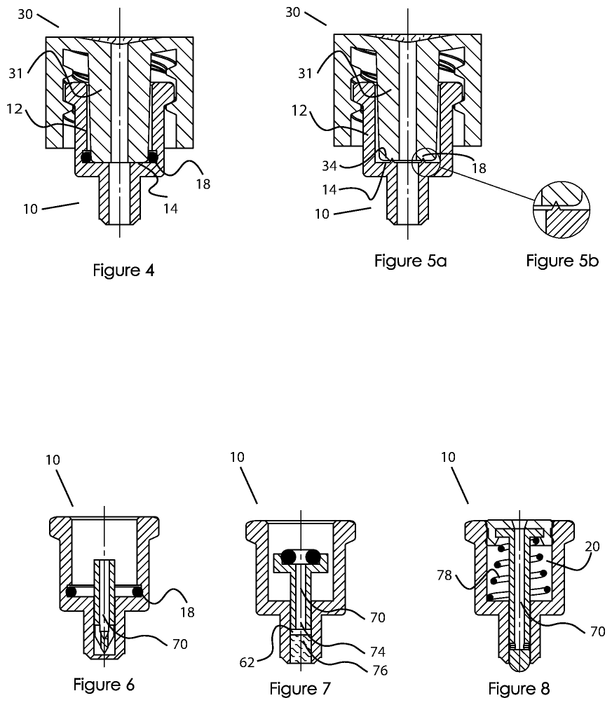

The seal 18 may form a fluid-tight closure at the interface of the distal engagement surface 14 of the female connector 10 and the tip surface 34 of the Luer protrusion 31. A fluid-tight closure may be formed by the interaction of the tip surface 34 and the seal 18. In some examples, the seal 18 may be configured to generally axially interact with the tip surface 34 to establish a fluid-tight seal. In other examples, the seal 18 may be configured to generally radially interact with the tip surface 34 to establish a fluid-tight seal (illustrated in FIG. 4). In these examples, the seal 18 may be of the same material as the receptacle 20 (illustrated in FIG. 5).

In other examples, the receptacle 20 may be a first material, and the seal 18 may be a second material more elastic than the first material. In some of these examples, the seal 18 maybe an O-ring. In other examples, the seal 18 may be a gasket. The seal 18 may be formed from neoprene, butyl rubber, silicone, EVA, or equivalent materials. In one example, the female connector is an injection-molded plastic component, and the seal is co-molded or over-molded from the same or different material from the female connector.

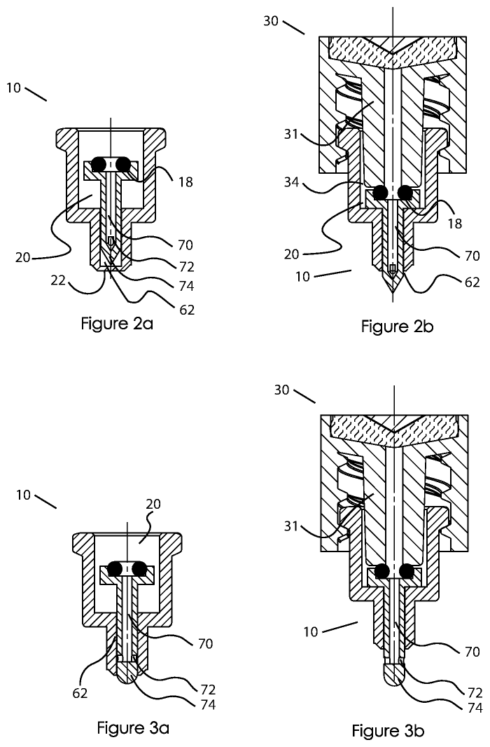

For purposes of brevity, elements presented in FIGS. 2a-12d may represent features similar or analogous to the like-named and/or like-numbered features presented in FIGS. 1a-1d.

FIG. 2a is a schematic illustration of an example female connector with a piercing member in a first position. The example female connector 10 may include a piercing element 70. The piercing element 70 may comprise a hollow channel spanning the long axis of the piercing element and an opening 72 at the distal end. The piercing element 70 may be in a first position. The first position may contain the piercing element 70 within the receptacle 20 of the female connector 10. The seal 18 may be joined with the piercing element 70. The receptacle 20 may contain a membrane 22 (e.g., a sealed membrane), which may be configured so as to block a fluid path 62 when the piercing element 70 is in the first position. The piercing element 70 may be movably disposed in the receptacle 20 such that it is in the first position prior to connecting to a male Luer connector 30 and is moved to a second position when the male Luer connector 30 is engaged. This second position is illustrated in FIG. 2b. FIG. 2b is a schematic illustration of an example female connector with a piercing member mated with a male Luer lock connector, wherein the piercing member is in a second position. In this implementation, the piercing element 70 opens the fluid path 62 by piercing through membrane 22 of the receptacle 20. The piercing element 70 may comprise a piercing end 74 with a sharp edge to facilitate piercing of the membrane. The piercing element 70 may be made of metal or rigid plastic material to facilitate piercing of the membrane. The seal 18 may be maintained in the second position and facilitates a fluid-tight seal between the tip surface 34 of the Luer protrusion 31 and the piercing element 70. In one implementation, the seal 18 may be molded over the piercing element. The operation of the piercing element 70 from the first position to a definitive location in the second position is enabled by avoiding the tapered interior walls of standard female Luer connector receptacles.

In one embodiment, the female connector 10 may be a molded component, and the membrane 22 is co-molded as a single component with the female Luer connector from the same or different materials. In one embodiment, the female connector 10 may be a molded component, and the piercing element 70 is co-molded as a single component with the female connector 10 from the same or different materials. In one embodiment, the membrane 22 may be made from a film, foil, sheet, or gasket and is joined with the female connector 10) by one of the means known in the art, such as heat welding or adhesives.

FIG. 3a is a schematic illustration of an example female connector comprising a plunger (e.g., a moveable plunger member) as a piercing element 70, wherein the interface between the female connector and the plunger form a valve, and the female is in a first configuration wherein the valve is closed. The plunger may include a flow path that partially defines the fluid path 62 or is in fluid communication with the fluid path 62. An example of a female connector 10 is shown wherein the fluid path 62 may be resealable. In this embodiment, the piercing end 74 of the piercing element 70 may block the fluid path 62 by sealing against it, serving the function of a membrane. An opening 72 to the channel within the piercing element 70 may be located behind the sealing surfaces of the piercing end 74, such that in the first position, there is no fluid through the fluid path 62.

In an implementation, the plunger is configured to actuate distally in response to an application of force by the tip of the male Luer lock connector to a top surface of the moveable plunger when the male Luer lock connector is brought into threaded engagement with the female connector. The actuation translates the plunger from a first position in which the combined fluid pathway is closed to a second position in which the combined fluid pathway is open.

FIG. 3b is a schematic illustration of an example female connector of FIG. 3a in a second configuration wherein the female connector is mated with a male Luer lock connector, and the valve is open. An example of a female connector 10 configured with a fluid path 62 that may be resealable is shown with the piercing element 70 in the second position after engaging the female connector 10 to the male Luer connector 30. The piercing element 70 has been advanced axially towards the distal end of the female connector 10, and the opening 72 of the piercing member is now outside the fluid path 62, permitting fluid communication through said fluid passageway. The fluid path 62 can be resealed by moving the piercing element 70 to the first position. In some embodiments (illustrated in FIG. 8), the piercing element 70 may be biased by a biasing member (e.g., a spring) towards the first position, such that disengaging the female connector 10 from the male Luer connector 30 may automatically move the piercing element 70 to the first position, resealing the fluid path 62. Implementations are contemplated in which the motion of the plunger irreversibly opens the combined fluid pathway.

FIG. 4 is a schematic illustration of an example female connector mated with a male Luerlock connector, wherein the seal interfaces with the peripheral region of the tip surface of the male Luer connector. An example of a female connector 10 engaged with a male Luer connector 30, wherein a seal 18 may be configured to interact radially with the Luer male connector, is shown. The interior wall 12 of the connector body 11 may comprise a circular notch in the region adjacent to the distal engagement surface 14 to accommodate a portion of the seal 18, therebyensuring that the seal 18 is positioned laterally to the tip surface 34.

FIG. 5a is a schematic illustration of an example female connector mated with a maleLuer lock connector, wherein the seal comprises a ridge feature embedded in the receptacle of the female connector. FIG. 5b is an enlarged view of an example female connector of FIG. 5a. An example of a female connector 10 is illustrated after the engagement with a male Luer connector 30. In this configuration, a seal 18 may be made of the same material as the connector body 11 and may comprise a ridge feature integral with the distal engagement surface 14. The tip surface 34 may interact with the seal 18 to form a fluid-tight interface. An enlargement of the seal 18 is shown in FIG. 5b. The seal 18 and ridge feature may be annular or circular to surround and provide a hermetic seal between the male Luer connector 30 and the female connector 10.

FIG. 6 is a schematic illustration of an example female connector comprising a piercing member and a stationary seal that is configured to interface with the larger diameter of the tip surface of a male Luer connector. An example is shown of a female connector 10 comprising a piercing element 70 and/or a seal 18 that may be stationary and/or that may be configured to interface with the larger diameter of a tip surface of a male Luer connector upon engagement.

FIG. 7 is a schematic illustration of an example female connector comprising a plug. The plug 76 may be movably and sealingly disposed inside of the fluid path 62. In this embodiment, a piercing end 74 of the piercing element 70 may be blunt (e.g., the piercing element may be a rod) and configured to push the plug 76 out of the fluid path 62 when the female connector 10 is engaged with a male Luer lock connector.

FIG. 8 is a schematic illustration of an example female connector comprising a biasing member 78 to form a valve. The biasing member 78 (e.g., a retraction spring) biases the plunger to the first position to keep the valve closed by default. An example of a female connector 10 comprising a biasing member 78 is shown. The biasing member 78 may be disposed in the receptacle 20, supporting the piercing element 70 and biasing it towards the first position. The female connector, therefore, effectively functions as a normally closed valve.

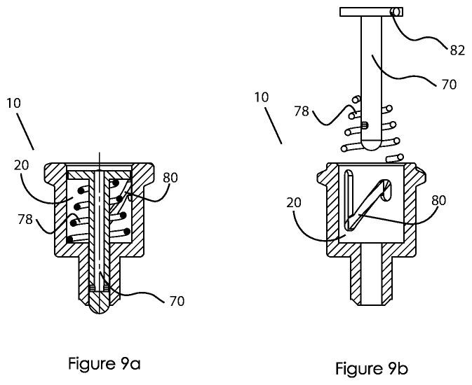

FIG. 9a is a schematic illustration of an example female connector 10 comprising an auto-disable mechanism. For example, the auto-disable mechanism includes a guide that allows the Luer protrusion to advance a first axial distance (e.g., along a longitudinal axis of the Luer protrusion) in a first advance and restricts the advancement of the Luer protrusion to a second distance shorter than the first distance in a second advance after the first advance. In this embodiment, the female connector 10 may include a biasing member 78 supporting the piercing element 70 and biasing it towards the first position. The receptacle 20 may include a guiding groove 80, and the piercing element 70 may comprise a pin (not visible), which mates with the groove 80. When the piercing element 70 is moved from the first position to the second position, the pin moves down the first leg of the guide in parallel with the long axis of the female connector 10. However, when the piercing element 70 is subsequently pushed in the direction of the first position by force exerted by the biasing member 78, the pin is forced into the second leg of the guide. This second leg leads to the third leg of the guide, which is significantly shorter than the first leg and limits the range of linear motion of the piercing element 70, effectively preventing it from being moved to the second position again. The auto-disable mechanism may help ensure that the female connector 10 cannot be used more than once. In an alternative implementation, the actuation in the second position returns the plunger to the first position (e.g., with or without a guide and pin assembly) upon disengagement of an advancing force.

FIG. 9b is an exploded view of an example female connector 10 comprising an auto-disable mechanism. In this implementation, the auto-disable mechanism is illustrated with the pin 82 visible.

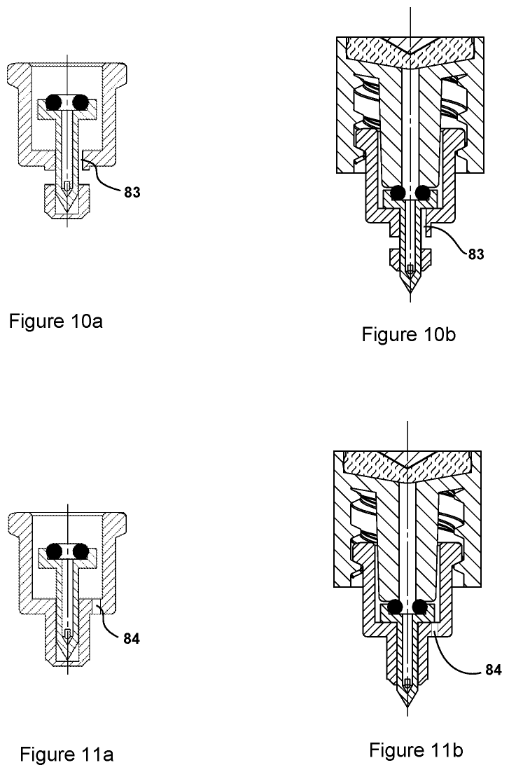

FIG. 10a is an illustration of an example female connector comprising an independent fluid venting pathway comprising an extended bore along the central shaft. An implementation of a female connector includes a vent 83 extending from the central bore, which forms an independent fluid pathway when interpenetrated by a male connector in the joined state. In this embodiment, the connector body and the body comprising the membrane 22 are depicted as separate molded components.

FIG. 10b illustrates the example female connector of FIG. 10a in the joined state with a corresponding male connector. FIG. 10b depicts the connector fully joined to a male connector, the piercing member penetrating the membrane 22, and the side bore open to the surrounding space. The vent may comprise any hole, perforation, bore, valve, frangible barrier, or equivalent secondary fluid path independent of the primary fluid path. The fluid connection of the path comprising the vent 83 may be augmented by one or more channels, tubes, crenulations, divots, or any method of preventing a tight seal between the female connector body and the penetrating element or analogous method of controlling the rate of fluid exchange in the joined state.

FIG. 11a is an illustration of an example female connector comprising an independent venting pathway comprising at least one separate bore in the connector body. In an implementation, the female connector includes a secondary fluid pathway 84 (e.g., a vent to relieve pressure) disposed as an independent bore which forms a separate secondary fluid pathway when interpenetrated by a male connector in the joined state. In this depiction, the connector body may include the membrane 22 of a packet (e.g., a flexible package containing a medicament as described herein) as a single molded component. As with the embodiment of FIG. 10a, the vent may comprise any hole, perforation, bore, valve, frangible barrier, or equivalent secondary fluid path independent of the primary fluid path.

FIG. 11b illustrates the female connector of FIG. 11a in the joined state with a corresponding male connector. FIG. 11b illustrates the joined or connected state of the female connector to a corresponding male connector, the extended male member bottoming out to form a fluid-tight seal against the piercing body, said piercing body penetrating the membrane 22 to establish a primary fluid pathway, the secondary fluid pathway 84 being simultaneously and separately comprised. Embodiments not comprising a piercing element may nevertheless comprise a venting pathway as described.

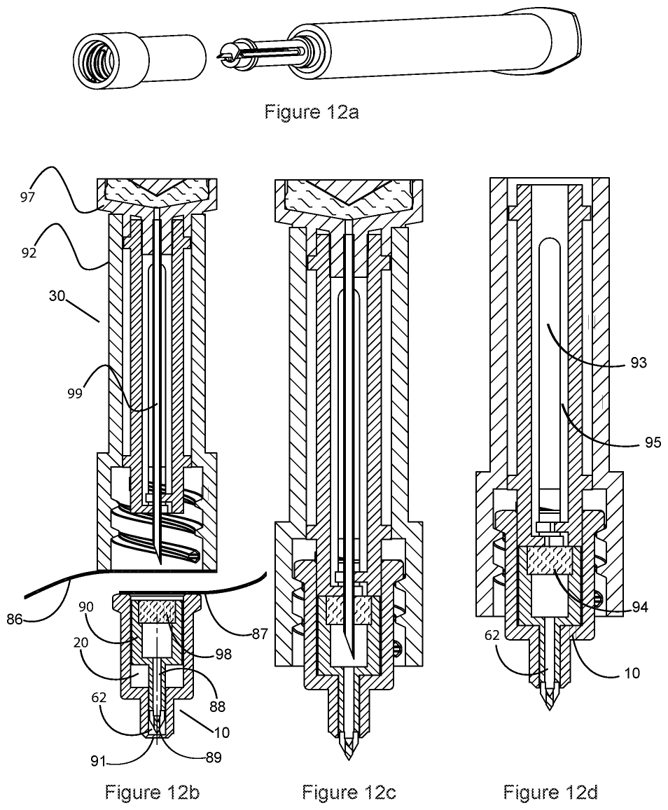

FIGS. 12a-d illustrate an implementation of a connector including a female connector 10 configured to establish fluid communication with a piercing element 97 (e.g., a needle or other conduits with a piercing tip). Specifically, FIG. 12a illustrates an external perspective view of an example medical connector. FIG. 12b illustrates a cross-sectional view of an example medical connector prior to engagement of a male Luer connector 30 and a female connector 10.

The male Luer connector 30 may include a detachable adapter 92 and a piercing element 97. The detachable adapter 92 may center the piercing element 97 and also may include a female thread configured to engage with the male thread of the female connector 10. The female connector 10 includes a body. The body includes a receptacle 20 at its first end and a fluid path 62 at its second end. The fluid path 62 is sealed by a pierceable membrane 91. The fluid path 62 may be connected to a tube, a pipe, a reservoir such as a vial or an infusion bag, or be open to the environment. A male thread is disposed on the body (e.g., on an exterior surface of the body) of the female connector 10 and is configured to engage with the female thread of the detachable adapter 92 when the needle is engaged with the female connector 10. The female connector 10 further comprises a plunger 90 having a first end comprising a septum 98 (or other sealed or resealable element) and a second end comprising a piercing member, and a flow conduit 88 therebetween. In other implementations (e.g., ones with or without the plunger 90), the detachable adapter 92 or the receptacle 20 may include a septum 98.

The piercing member 89 of the moveable plunger is configured to pierce the pierceable membrane 91 disposed at the distal end of the fluid path 62 (e.g., the end of a combined fluid pathway including the fluid path 62 of the female connector 10, the flow conduit 88 of the plunger 90, and/or the flow channel 99 of the piercing element 97) when the piercing element 97 is engaged with the female connector 10. FIG. 12a illustrates the implementation prior to engaging the piercing element 97 with the female connector 10. A first peelable foil 86 covers the end of the detachable adapter 92 to maintain the piercing element 97 in an aseptic environment. A second peelable foil 87 covers the end of the female connector 10 to keep the fluid path 62, the plunger 90 and/or receptacle 20 sterile.

FIG. 12c illustrates a cross-sectional view of an example medical connector after engagement of a male Luer connector 30 and a female connector 10. The piercing element 97 may engage the female connector 10 to establish a combined fluid pathway between the flow channel 99 of the piercing element 97 and the fluid path 62 of the female connector 10 (e.g., via the flow conduit 88 of the plunger 90). The piercing element 97 pierces through the septum 98 to establish fluid communication with the fluid path 62 of the female connector 10. The piercing action of the piercing element 97 upon threaded engagement to the female connector 10 may apply a force sufficient to advance the plunger 90 distally within a receptacle 20 of the female connector 10. The detachable adapter 92 moves the plunger 90 forward as the threads of the connectors mutually engage, and the plunger pierces through the pierceable membrane 91. The piercing element 97 remains protected by the detachable adapter 92 during the engagement process. The implementation may be configured such that a substance may be drawn from a first location such as collapsible container, vial, IV bag, etc. into a storage or dispensing device such as a syringe through the combined fluid pathway of the mutually engaged male Luer connector 30, the female connector 10, and the piercing element 97.

FIG. 12d illustrates a cross-sectional view of an example medical connector after engagement of a male Luer connector 30 and a female connector 10 and after a piercing element 97 is removed. For example. FIG. 12d illustrates an implementation of the connector of FIGS. 12a-c when the piercing element 97 is removed. The detachable adapter 92 remains connected to the female connector 10. The piercing member 89 may be exposed and prepared for insertion into a subject to administer a withdrawn beneficial agent or other substance. The piercing member 89 may be inserted into a storage volume such as a vial. IV bag, infusion set or other storage volume to add the withdrawn constituents to its stored volume or contents. The plunger 90 may open the conduit as described herein, or by equivalent techniques.

With respect to the implementations of the male Luer connectors and female connectors (collectively, the connectors) described herein, it should be appreciated that the connectors can be utilized for connecting a flexible package for containing and selectively administering the contents of the package by establishing fluid communication with an administration device via one or more of the connectors. For example, either of the connectors can be used as elements of the flexible package itself (e.g., a fitment) or of an element configured to interface with the flexible package (e.g., a syringe, auto-injector, intravenous line, or another administration device). Examples of packages and fitments can be found in U.S. Pat. No. 9,820,913, which is incorporated herein by reference in its entirety.

An example small bore medical device connector is provided. The small bore medica device connector includes a female connector comprising a fluid path and a receptacle configured to accept a male Luer lock connector protrusion extending from a male Luer lock connector, the female connector configured for threaded engagement to the male Luer lock connector. The male Luer lock connector protrusion of the male Luer lock connector includes a standard Luer tapered protrusion extending continuously between a proximal portion and a distal portion of the male Luer lock connector protrusion and a flow channel therewithin. The male Luer lock connector terminates at a tip disposed at the distal portion, the tip comprising an opening to the flow channel. When the female connector is threadedly engaged with the male Luer lock connector, a distal aspect of the tip of the male Luer lock connector protrusion contactingly engages a distal engagement surface in the receptacle of the female connector to establish a fluid-tight seal and to establish fluid communication through a combined fluid pathway comprising the fluid path of the female connector and the flow channel of the male Luer lock connector.

Another example small bore medical device connector of any preceding connector is provided, wherein the female connector includes an external threading engagement portion positioned externally of the receptacle, and the male Luer lock connector includes an interior threading portion facing internally towards the male Luer lock connector protrusion, wherein the threadable engagement includes engagement of the external threading engagement portion with the interior threading portion.

Another example small bore medical device connector of any preceding connector is provided, wherein the external threading engagement portion includes radial protrusions extending outward at a proximal end of the receptacle.

Another example small bore medical device connector of any preceding connector is provided, wherein the distal engagement surface in the receptacle is substantially perpendicular to a direction of axial advancement of the male Luer lock connector protrusion along a longitudinal axis of the male Luer lock connector protrusion during the threadable engagement.

Another example small bore medical device connector of any preceding connector is provided, wherein the receptacle comprises a recessed volume defined by a wall and a base surface, the wall being substantially parallel to the standard Luer tapered protrusion of the male Luer lock connector when in the threaded engagement, the base surface being substantially parallel to the distal aspect of the tip when the female connector and the male Luer lock connector are engaged, the base surface comprising the distal engagement surface.

Another example small bore medical device connector of any preceding connector is provided, wherein a distance remains between the wall and the male Luer lock connector protrusion at all axial positions of a longitudinal axis of the male Luer lock connector protrusion when the distal aspect of the tip contactingly engages the distal engagement surface.

Another example small bore medical device connector of any preceding connector is provided, wherein a geometry of the receptacle is configured such that when the male Luer lock connector is engaged, the distal aspect of the tip of the male Luer lock connector bottoms out against the distal engagement surface.

Another example small bore medical device connector of any preceding connector is provided, further comprising a sealing member disposed in the receptacle, the sealing member configured to establish a fluid-tight seal when the distal aspect of the tip bottoms out against the distal engagement surface, wherein a geometry of the receptacle is adapted to form the fluid-tight seal between the distal aspect of the tip and the receptacle using the sealing member.

Another example small bore medical device connector of any preceding connector is provided, wherein the sealing member comprises an over-molded feature of the distal engagement surface.

Another example small bore medical device connector of any preceding connector is provided, further including a sealing member disposed in the receptacle, wherein the sealing member includes the distal engagement surface.

Another example small bore medical device connector of any preceding connector is provided, wherein the receptacle comprises a recessed volume defined by a wall, the wall being substantially parallel to the standard Luer tapered protrusion of the male Luer lock connector when in threaded engagement with the female connector, wherein a tapered surface of the standard Luer tapered protrusion of the male Luer lock connector does not engage the wall to form a seal.

Another example small bore medical device connector of any preceding connector is provided, wherein when the tip of the male Luer lock connector bottoms out at the distal engagement surface, a fluid passageway is established between the standard Luer tapered protrusion of the male Luer lock connector and a wall of the receptacle.

Another example small bore medical device connector of any preceding connector is provided, the male Luer lock connector comprising a standard male Luer lock connector per ISO Standard 80369 comprising a Luer lock thread and a Luer protrusion comprising a 6% tapered surface.

Another example small bore medical device connector of any preceding connector is provided, further including a moveable plunger member configured to actuate distally in response to an application of force by the tip of the male Luer lock connector to a top surface of the moveable plunger member when the male Luer lock connector is brought into threaded engagement with the female connector, the actuation translating the moveable plunger member from a first position in which the combined fluid pathway is closed to a second position in which the combined fluid pathway is open.

Another example small bore medical device connector of any preceding connector is provided, the moveable plunger member further comprising a flow path, wherein the distal engagement surface includes the top surface disposed within the receptacle.

Another example small bore medical device connector of any preceding connector is provided, wherein, in the second position, the flow path comprises a section of the combined fluid pathway.

Another example small bore medical device connector of any preceding connector is provided, wherein a distal opening of the flow path is sealed in the first position and open in the second position.

Another example small bore medical device connector of any preceding connector is provided, the moveable plunger member further comprising a rod generally parallel to the fluid path, and wherein distal actuation of the moveable plunger member translates the rod through a sealed membrane causing the sealed membrane to rupture.

Another example small bore medical device connector of any preceding connector is provided, wherein the rod is partially accommodated in the fluid path of the receptacle of the female connector.

Another example small bore medical device connector of any preceding connector is provided, the moveable plunger member further comprising a rod generally parallel to the fluid path, and wherein distal actuation of the moveable plunger member translates the rod to displace a plug and open the combined fluid pathway.

Another example small bore medical device connector of any preceding connector is provided, wherein actuation into the second position irreversibly opens the combined fluid pathway.

Another example small bore medical device connector of any preceding connector is provided, further including a biasing member disposed to return the moveable plunger member to the first position upon disengagement of the male Luer lock connector from the female connector.

Another example small bore medical device connector of any preceding connector is provided, wherein the biasing member is at least partially positioned between the male Luer lock connector protrusion and the receptacle when the female connector is threadedly engaged with the male Luer lock connector.

Another example small bore medical device connector of any preceding connector is provided, further including a piercing element movably disposed in the receptacle such that it is in a first position prior to the threaded engagement and is translated to a second position when in the threaded engagement.

Another example small bore medical device connector of any preceding connector is provided, further including a vent comprising a secondary fluid pathway not in fluid communication with the combined fluid pathway when the female connector and male Luer lock connector are in threaded engagement.

Another example small bore medical device connector of any preceding connector is provided, wherein the receptacle is less than 7.5 mm deep from a proximal end of the receptacle to the distal engagement surface.

Another example small bore medical device connector of any preceding connector is provided, wherein a maximum penetration depth of the male Luer lock connector protrusion into the receptacle is less than 7.5 mm.

Another example small bore medical device connector of any preceding connector is provided, wherein a diameter of the receptacle is more than 4.477 mm.

Another example small bore medical device connector of any preceding connector is provided, further comprising an auto-disable mechanism including a guide to allow the male Luer lock connector protrusion to advance a first axial distance in a first advance and restrict the advancement of the male Luer lock connector protrusion to a second axial distance shorter than the first axial distance in a second advance after the first advance.

Reference throughout the specification to “one example.” “another example.” “an example.” and so forth means that a particular element (e.g., feature, structure, and/or characteristic) described in connection with the example is included in at least one example described herein, and may or may not be present in other examples. In addition, it is to be understood that the described elements for any example may be combined in any suitable manner in various examples unless the context clearly indicates otherwise.

In describing and claiming the examples disclosed herein, the singular forms “a,” “an,” and “the” include plural referents unless the context clearly indicates otherwise.

From the foregoing, it will be appreciated that specific embodiments of the invention have been described herein for purposes of illustration but that various modifications may be made without deviating from the scope of the invention. Although the technology has been described in language that is specific to certain structures and materials, it is to be understood that the invention defined in the appended claims is not necessarily limited to the specific structures and materials described. Rather, the specific aspects are described as forms of implementing the claimed invention. Because many embodiments of the invention can be practiced without departing from the spirit and scope of the invention, the invention resides and is limited solely by the claims hereinafter appended.

The logical operations making up implementations of the technology described herein may be referred to variously as operations, steps, objects, or modules. Furthermore, it should be understood that logical operations may be performed in any order, adding or omitting operations as desired, regardless of whether operations are labeled or identified as optional, unless explicitly claimed otherwise or a specific order is inherently necessitated by the claim language.

While this specification contains many specific implementation details, these should not be construed as limitations on the scope of any technologies or of what may be claimed but rather as descriptions of features specific to particular implementations of the particular described technology. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can, in some cases, be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order or that all illustrated operations be performed to achieve desirable results. Furthermore, it should be understood that logical operations may be performed in any order, adding or omitting operations as desired, regardless of whether operations are labeled or identified as optional, unless explicitly claimed otherwise or a specific order is inherently necessitated by the claim language. In some cases, the actions recited in the claims can be performed in a different order and still achieve desirable results. In certain circumstances, multitasking and parallel processing may be advantageous. The logical operations making up implementations of the technology described herein may be referred to variously as operations, steps, objects, or modules.

Moreover, the separation of various system components in the implementations described above should not be understood as requiring such separation in all implementations, and it should be understood that the described program components and systems can generally be integrated together in a single software product or packaged into multiple software products. Thus, particular implementations of the subject matter have been described. Other implementations are within the scope of the following claims. Nevertheless, it will be understood that various modifications can be made without departing from the spirit and scope of the recited claims.

As used herein, terms such as “substantially.” “about.” “approximately.” or other terms of relative degree are interpreted as a person skilled in the art would interpret the terms and/or amount to a magnitude of variability of one or more of 1%, 2%, 3%, 4%, 5%. 6%, 7%, 8%, 9%, 10%, 11%, 12%, 13%, 14%, or 15% of a metric relative to the quantitative or qualitative feature described. For example, a term of relative degree applied to orthogonality suggests an angle may have a magnitude of variability relative to a right angle. When values are presented herein for particular features and/or a magnitude of variability, ranges above, ranges below, and ranges between the values are contemplated. It is to be understood that the ranges provided herein include the stated range and any value or sub-range within the stated range, as if the value(s) or sub-range(s) within the stated range were explicitly recited. For example, a range from about 1 millimeter to about 6 millimeters should be interpreted to include not only the explicitly recited limits of from about 1 millimeter to about 6 millimeters but also to include individual values, such as about 1.6 millimeters, about 3.89 millimeters, about 5.22 millimeters, etc., and sub-ranges such as from about 2.1 millimeters to about 4.9 millimeters, from about 3.61 millimeters to about 5.9 millimeters, etc.

1. A small bore medical device connector comprising:
a female connector comprising a fluid path and a receptacle configured to accept a male Luer lock connector protrusion extending from a male Luer lock connector, the female connector configured for threaded engagement to the male Luer lock connector,
wherein the male Luer lock connector protrusion of the male Luer lock connector comprises:
a standard Luer tapered protrusion extending continuously between a proximal portion and a distal portion of the male Luer lock connector protrusion; and
a flow channel therewithin,
wherein the male Luer lock connector terminates at a tip disposed at the distal portion, the tip comprising an opening to the flow channel, and
wherein, when the female connector is threadedly engaged with the male Luer lock connector, a distal aspect of the tip of the male Luer lock connector protrusion contactingly engages a distal engagement surface in the receptacle of the female connector to establish a fluid-tight seal and to establish fluid communication through a combined fluid pathway comprising the fluid path of the female connector and the flow channel of the male Luer lock connector.

2. The small bore medical device connector of claim 1, wherein the female connector includes an external threading engagement portion positioned externally of the receptacle, and the male Luer lock connector includes an interior threading portion facing internally towards the male Luer lock connector protrusion, wherein the threadable engagement includes engagement of the external threading engagement portion with the interior threading portion.

3. (canceled)

4. The small bore medical device connector of claim 1, wherein the distal engagement surface in the receptacle is substantially perpendicular to a direction of axial advancement of the male Luer lock connector protrusion along a longitudinal axis of the male Luer lock connector protrusion during the threaded engagement.

5. The small bore medical device connector of claim 1, wherein the receptacle comprises a recessed volume defined by a wall and a base surface, the wall being substantially parallel to the standard Luer tapered protrusion of the male Luer lock connector when in the threaded engagement, the base surface being substantially parallel to the distal aspect of the tip when the female connector and the male Luer lock connector are engaged, the base surface comprising the distal engagement surface.

6. The small bore medical device connector of claim 5, wherein a distance remains between the wall and the male Luer lock connector protrusion at all axial positions of a longitudinal axis of the male Luer lock connector protrusion when the distal aspect of the tip contactingly engages the distal engagement surface.

7. The small bore medical device connector of claim 1, wherein a geometry of the receptacle is configured such that when the male Luer lock connector is engaged, the distal aspect of the tip of the male Luer lock connector bottoms out against the distal engagement surface.

8. The small bore medical device connector of claim 1, further comprising a sealing member disposed in the receptacle, the sealing member configured to establish a fluid-tight seal when the distal aspect of the tip bottoms out against the distal engagement surface, wherein a geometry of the receptacle is adapted to form the fluid-tight seal between the distal aspect of the tip and the receptacle using the sealing member.

9. (canceled)

10. The small bore medical device connector of claim 1, further comprising a sealing member disposed in the receptacle, wherein the sealing member includes the distal engagement surface.

11. The small bore medical device connector of claim 1, wherein the receptacle comprises a recessed volume defined by a wall, the wall being substantially parallel to the standard Luer tapered protrusion of the male Luer lock connector when in threaded engagement with the female connector, wherein a tapered surface of the standard Luer tapered protrusion of the male Luer lock connector does not engage the wall to form a seal.

12. The small bore medical device connector of claim 1, wherein when the tip of the male Luer lock connector bottoms out at the distal engagement surface, a fluid passageway is established between the standard Luer tapered protrusion of the male Luer lock connector and a wall of the receptacle.

13. (canceled)

14. The small bore medical device connector of claim 1, further comprising a moveable plunger member configured to actuate distally in response to an application of force by the tip of the male Luer lock connector to a top surface of the moveable plunger member when the male Luer lock connector is brought into threaded engagement with the female connector, the actuation translating the moveable plunger member from a first position in which the combined fluid pathway is closed to a second position in which the combined fluid pathway is open.

15. (canceled)

16. The small bore medical device connector of claim 11, wherein, in the second position, the flow path comprises a section of the combined fluid pathway.

17. The small bore medical device connector of claim 11, wherein a distal opening of the flow path is sealed in the first position and open in the second position.

18. The small bore medical device connector of claim 11, the moveable plunger member further comprising a rod generally parallel to the fluid path, and wherein distal actuation of the moveable plunger member translates the rod through a sealed membrane causing the sealed membrane to rupture.

19. (canceled)

20. The small bore medical device connector of claim 11, the moveable plunger member further comprising a rod generally parallel to the fluid path, and wherein distal actuation of the moveable plunger member translates the rod to displace a plug and open the combined fluid pathway.

21. (canceled)

22. The small bore medical device connector of claim 14, further comprising a biasing member disposed to return the moveable plunger member to the first position upon disengagement of the male Luer lock connector from the female connector.

23. The small bore medical device connector of claim 22, wherein the biasing member is at least partially positioned between the male Luer lock connector protrusion and the receptacle when the female connector is threadedly engaged with the male Luer lock connector.

24. The small bore medical device connector of claim 1, further comprising a piercing element movably disposed in the receptacle such that it is in a first position prior to the threaded engagement and is translated to a second position when in the threaded engagement.

25. The small bore medical device connector of claim 1, further comprising a vent comprising a secondary fluid pathway not in fluid communication with the combined fluid pathway when the female connector and male Luer lock connector are in threaded engagement.

26. (canceled)

27. (canceled)

28. (canceled)

29. The small bore medical device connector of claim 1, further comprising an auto-disable mechanism including a guide to allow the male Luer lock connector protrusion to advance a first axial distance in a first advance and restrict the advancement of the male Luer lock connector protrusion to a second axial distance shorter than the first axial distance in a second advance after the first advance.
